(19) United States
(12) Patent Application Publication
(10) Pub. No.: US 2003/0199814 A1
(43) Pub. Date: Oct. 23, 2003

(54) Multi-component ampule

(76) Inventors: J. Stuart Parsons, Redondo Beach, CA (US); Kathleen Parsons, Redondo Beach, CA (US)

Correspondence Address: CHRISTIE, PARKER & HALE, LLP, P.O. BOX 7068, PASADENA, CA, 91109-7068, US

(21) Appl. No.: 10/422,419

(22) Filed: Apr. 23, 2003

Related U.S. Application Data
(63) Continuation-in-part of application No. 10/215,856, filed on Aug. 8, 2002.
(60) Provisional application No. 60/374,461, filed on Apr. 19, 2002.

(51) Int. Cl. A61M 5/30

(52) U.S. Cl. 604/68; 604/72

(57) ABSTRACT

Multi-component ampules for use with re-useable and disposable jet injectors are described which have primary and secondary packaging components. The primary packaging component includes an inner glass cylinder, an elastomeric diaphragm, and an elastomeric plunger. The secondary packaging components include a plastic outer shell and a plastic adapter. The primary packaging components allow medications and injectable suspensions to be stored for prolonged periods while the secondary packaging components provide structural integrity and adaptability for the ampule. Optionally, thermoplastics having long term storage capability may also be used for the primary packaging components. Exemplary methods for using the jet syringe assembly and of making the same are also disclosed.

### CROSS-REFERENCE TO RELATED APPLICATION

This is a continuation-in-part application of utility application Ser. No. 10/215,856, filed on Aug. 8, 2002, which claims the benefit of provisional application No. 60/374,461, filed on Apr. 19, 2002, both of which are entitled “Multi-Component Ampule”, their contents are expressly incorporated herein by reference.

Ampules discussed herein generally relate to ampules that are used for long term storage of medications and the like, and more specifically to multi-component ampules that are made from different materials and their applications.

### BACKGROUND

Jet injection devices are well known in the art for administering intramuscular and subcutaneous medications without needles. Examples of hypodermic jet injectors are described in U.S. Pat. Nos. 5,499,972; 5,569,189; and 5,704,911, their contents are hereby expressly incorporated herein by reference.

In general, these patents disclose a hypodermic jet injector device that has an ampule for holding liquid medication and a jet injector for receiving the ampule and for injecting medication contained within the ampule subcutaneously without a needle. The ampule is generally a single integral component made from a thermoplastic material that has a nozzle on one end for discharging medication, the discharge end, and an opening on the other end for securing the ampule to a jet injector, the inlet end. The inlet end further includes a connectable end, such as an end with external or internal threads or a sleeve for mechanically coupling to the jet injector. The ampule's physical characteristics such as wall thickness and diameter are determined in part by the desired delivery dosage, the plunger type, the nozzle size, and the operating pressure for delivering the medication subcutaneously without a needle.

The jet injector includes a metallic cylinder enclosed on one end, such as with a plug or cap, and open on the other end for receiving the ampule. Within the cylinder, the components of the jet injector generally include a spring, a piston, a shaft, a plunger, and a trigger. The jet injector device operates by cocking or compressing the spring, which is in mechanical communication with the piston. The trigger is used to set off the spring, which drives the piston, which then drives the shaft, and which then drives the plunger into the medication to discharge the medication out the nozzle at the distal end of the ampule. A typical operating pressure for a jet injector device to deliver medication subcutaneously without a needle is in the range of about 3,000 to 3,500 psi at the nozzle, with a much higher pressure range of about 5,000 to 6,000 psi developed during the initial thrust of the piston. Thus, a suitable ampule for use with the jet injector is one that is capable of handling the aforementioned pressure range.

Subsequent to discharging the medication, the ampule, plunger, and shaft may be separated from the jet injector and be disposed of. The injector, however, can be re-used by resetting the spring, as disclosed in the '911 patent. A new ampule, plunger, and shaft may then be connected to the jet injector by threading the ampule into the receiving end of the cylinder of the jet injector.

Another hypodermic jet injector example is disclosed in Ser. No. 09/751,525 filed Dec. 29, 2000 and entitled “Low Cost Disposable Needleless Injector System for Variable and Fixed Dose Applications”, the content of which is incorporated herein by reference. The '525 Ser. No. discloses a jet injector assembly designed for low cost production and for disposability after a single use. The disposable jet injector assembly generally comprises an ampule threadedly or permanently attached to a jet injector. Within the jet injector, the device includes a spring that is in dynamic communication with a shaft and a piston. The shaft is coaxially disposed within the piston and is moveable or slidable within the piston even when the piston is in a cocked position. However, the disposable jet injector generally comes pre-cocked or pre-set in a package from the factory and only requires filling the ampule with medication at the point of injection. The shaft has a length such that a portion of the shaft extends out from the jet injector housing to facilitate filling the ampule, by grasping and moving the extended shaft portion.

The ampule is threadedly or permanently fixed to the jet injector by adhesive, heat or ultrasonic welding. The shaft, via the extension, allows medication to be drawn into the ampule when it is retracted from a first position to a second position, which creates a vacuum in the ampule to thereby draw in medication. The injector assembly is used by placing the discharge nozzle next to the skin and then firing the trigger, as discussed above with reference to the re-useable jet injector model.

Although both the disposable and the re-useable jet injector assemblies are effective, reliable, and economical, they suffer from at least one shortcoming. Among other things, they utilize thermoplastic (“plastic”) ampules for manufacturability and for high-pressure compatibility. Plastic is relatively ductile, has a low modulus of elasticity, is highly impact resistant, and components that are made from plastic are relatively easy to fabricate. However, ampules produced from plastic are generally not suitable for long term storage of medications, injectable suspensions, or the like. This is because certain ingredients that are added during the fabrication process of the plastic ampules can leach into the medication. Certain drugs or components of the drugs are also known to bind with the plastic or be absorbed by the plastic. Oxidation, degradation, and/or precipitation of the medication are also known to occur with prolonged storage of the medication in plastic ampules. It is also possible for a component of the drug to migrate through the walls of the plastic ampule, and oxygen, carbon dioxide, or other gasses may pass through the plastic into the drug. Hence, plastic ampules are generally not FDA approved for long term storage of medications and injectable suspensions.

ASTM Type I Class A and United States Pharmacopeia (USP) Type I glass are FDA approved glass for prolonged storage of medications and injectable suspensions. However, glass has a high modulus of elasticity and is highly brittle. Thus, if an ampule is made from glass, the ampule has to be sufficiently thick in order to have the hoop strength necessary to accommodate an operating pressure of about 3,000 to 3,500 psi, and about 5,000 to 6,000 psi at the start of the injection cycle. A glass ampule that is capable of withstanding this pressure range, however, can be expensive, unsightly, and undesirable when used in connection with the jet injectors described.

There is therefore a need for a multi-component ampule that is useable with conventional jet injectors, that is capable of long term storage of medications and injectable suspensions, and that uses FDA approved materials. Additionally, there is also a need for a method of using the multi-component ampule with the conventional jet injectors.

### SUMMARY OF THE INVENTION

The present invention specifically addresses and alleviates the above-mentioned deficiencies associated with the prior art assemblies. More particularly, the present invention comprises a multi-component ampule that has materials capable of storing injectable fluids, such as medications, therapies or vaccines, for prolonged periods.

Exemplary multi-component ampules provided in accordance with practice of the present invention include ampules that have a primary packaging component and a secondary packaging component. Broadly speaking, the multi-component ampule is characterized by an inner cylinder of a first material, an outer shell of a second material, and a diaphragm of a third material. Optionally, the diaphragm may be integrally molded to the inner cylinder and including a nozzle comprising an orifice.

Broadly speaking, a jet injector assembly comprising an ampule having an inner shell and an outer shell attached to a spring injector, wherein the spring injector comprising a housing comprising a plug having a passage engaged to an end of the housing, a shaft coaxially disposed with a piston and with a spring and partially extending through the passage of the plug, and wherein the spring is in abutting relationship with the piston and the plug.

Another aspect of the present invention includes a jet injector assembly comprising an ampule comprising an inner shell having a first closed end comprising an integrally molded nozzle and an open second end comprising female threads, wherein the inner shell is made from a first thermoplastic material; and an outer shell co-molded to the inner shell having first and second open ends, wherein the outer shell is made from a second thermoplastic material. A plunger in dynamic sealing communication with an interior cavity of the inner shell may be included, wherein the plunger is adapted to discharge contents from within the interior cavity of the inner shell out of the integrally molded nozzle when moved from a first position to a second position within the inner shell. Optionally a cap cover or a foil innerseal attached to a distal end of the inner shell may be provided.

Another aspect of the present invention includes a jet injector assembly comprising an ampule attached to a jet injector; wherein the ampule comprises an inner shell made from a first material having a first end, a second end, and an interior cavity; a diaphragm made from a second material positioned in the interior cavity of the inner shell adjacent the first end; and an outer shell made from a third material coaxially disposed over the inner shell.

It is understood that changes in the specific structure shown and described may be made within the scope of the claims without departing from the spirit of the present invention.

### BRIEF DESCRIPTION OF THE DRAWINGS

These and other features, aspects and advantages of the present invention will be more fully understood when considered with respect to the following detailed description, appended claims and accompanying drawings, wherein:

FIG. 1 is a semi-schematic cross-sectional view of an exemplary multi-component ampule provided in accordance with practice of the present invention;

FIG. 2 is a semi-schematic cross-sectional view of an outer shell shown separated from the multi-component ampule of FIG. 1;

FIG. 3 is a semi-schematic cross-sectional view of a diaphragm shown separated from the multi-component ampule of FIG. 1;

FIG. 4 is a semi-schematic cross-sectional view of an inner cylinder shown separated from the multi-component ampule of FIG. 1

FIG. 5 is a semi-schematic end view of a cap cover shown separated from the multi-component ampule of FIG. 1;

FIG. 6 is a semi-schematic cross-sectional view of the cap cover of FIG. 5 taken line 6-6;

FIG. 7 is a semi-schematic end view of a cap seal shown separated from the multi-component ampule of FIG. 1;

FIG. 8 is a semi-schematic cross-sectional view of the cap seal of FIG. 7 taken at line 8-8;

FIG. 9 is a semi-schematic cross-sectional view of a threaded nipple shown separated from the multi-component ampule of FIG. 1;

FIG. 10 is a semi-schematic side view of an exemplary plunger provided in accordance with practice of the present invention;

FIG. 11 is a semi-schematic side view of an alternative plunger provided in accordance with practice of the present invention;

FIG. 12 is a semi-schematic side view of an exemplary shaft coupled to yet another alternative plunger provided in accordance with practice of the present invention

FIG. 13 is a semi-schematic cross-sectional view of the ampule shown in FIG. 1 in a filled state;

FIG. 14 is a semi-schematic cross-sectional view of the ampule shown in FIG. 13 with a protective housing provided in accordance with practice of the present invention covering a portion of the shaft;

FIG. 15 is a semi-schematic cross-sectional view of an exemplary reuseable jet injector assembly with the ampule of FIG. 1 provided in accordance with practice of the present invention;

FIG. 16 is a semi-schematic cross-sectional view of the re-useable jet injector assembly of FIG. 15 in a spent or discharged state;

FIG. 17 is a semi-schematic cross-sectional view of an exemplary disposable jet injector assembly with the ampule of FIG. 1 provided in accordance with practice of the present invention;

FIG. 18 is a partial semi-schematic cross-sectional view of the disposable jet injector assembly of FIG. 17 with modified attachments between the jet injector and the ampule;

FIG. 19 is a semi-schematic cross-sectional side view a multi-component ampule that has a recessed section on the distal end of the outer shell with a protective housing provided in accordance with practice of the present invention;

FIG. 20 is a semi-schematic cross-sectional side view of the outer shell of FIG. 19;

FIG. 21 is a semi-schematic cross-sectional side view of a disposable jet injector assembly having a multi-component ampule threaded to a jet injector; and

FIG. 22 is a semi-schematic partial cross-sectional side view of an alternative jet injector assembly provided in accordance with aspects of the present invention, which comprises a housing having a plug and an ampule having rounded contours;

FIG. 23 is a semi-schematic partial cross-sectional side view of an alternative diaphragm provided in accordance with aspects of the present invention;

FIG. 24 is a semi-schematic partial cross-sectional side view of an alternative outer shell provided in accordance with aspects of the present invention;

FIG. 25 is a semi-schematic cross-section side view of an alternative piston provided in accordance with aspects of the present invention;

FIGS. 26a-26c depict various views of an alternative jet syringe housing provided in accordance with aspects of the present invention;

FIGS. 26a-26b depict various views of a trigger safety slide provided in accordance with aspects of the present invention;

FIG. 28 is a semi-schematic partial cut-away side view of a trigger provided in accordance with aspects of the present invention;

FIG. 29 is a semi-schematic cross-sectional side view of an alternative ampule provided in accordance with aspects of the present invention; which is shown attached to a threaded nipple and to a jet syringe (partially shown).

### DETAILED DESCRIPTION

The detailed description set forth below in connection with the appended drawings is intended as a description of the presently preferred embodiments of the multi-component ampule in accordance with the present invention and is not intended to represent the only forms in which the present invention may be constructed or utilized. The description sets forth the features and the steps for constructing and using the multi-component ampule of the present invention in connection with the illustrated embodiments. It is to be understood, however, that the same or equivalent functions and structures may be accomplished by different embodiments that are also intended to be encompassed within the spirit and scope of the invention. Also, as denoted elsewhere herein, like element numbers are intended to indicate like or similar elements or features.

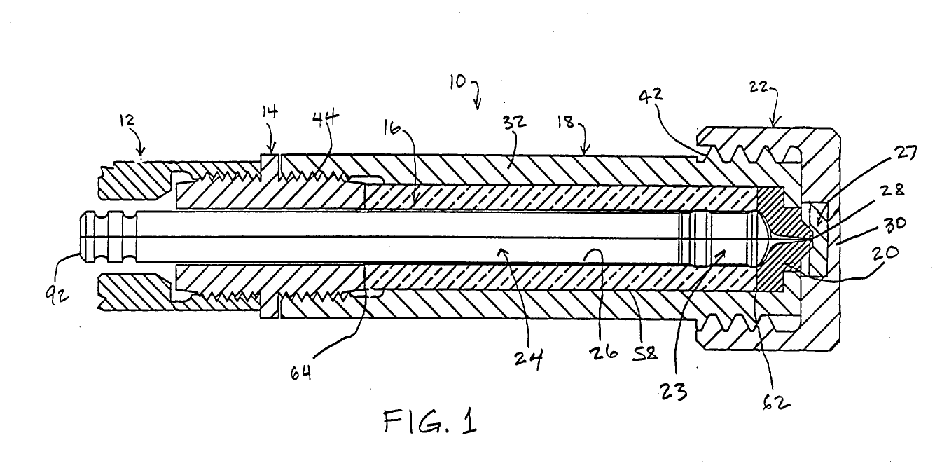

Referring now to FIG. 1, there is shown a multi-component ampule (herein “ampule”) provided in accordance with practice of the present invention, which is generally designated 10. The ampule 10 shown is connected to a jet injector 12 (partially shown), which may be a disposable or a reusable type jet injector, via a threaded nipple 14. Broadly speaking, the ampule 10 shown includes an inner cylinder 16, an outer shell 18, a diaphragm 20, and a cap cover 22, herein collectively referred to as components. Together, the components provide long-term storage capability and allow the ampule 10 to be used in high-pressure applications, such as those discussed further below.

In an exemplary embodiment, the inner cylinder 16 is made from an FDA approved glass for packaging human drugs and biological products, which currently includes ASTM Type I, Class A, and United States Pharmacopeia (USP) Type I glass. However, materials other than glass are contemplated to be used for the inner cylinder 16 provided they meet FDA requirements for long term storage of injectable human drugs. These alternative materials may include Type II glass, certain plastic, and certain elastomeric components which comply with USP Elastomeric Closures for Injections requirements. Such alternative materials may include polypropylene and silicone polymer.

Materials for the outer shell 18 and the cap cover 22 are made from plastic such as polycarbonate, acrylonitrile-butadiene, or the like. Materials for the diaphragm 20 and the plunger 23 are preferably made from an elastomer such as silicone rubber, fluroelastomer and the like. As will be appreciated, the outer shell 18 is preferably transparent so that the contents may be observed during shipment and during use, such as before an injection. Together the diaphragm 20, the plunger 23, and the inner cylinder 16 make up part of the primary packaging containment while the outer shell 18 and the cap cover 22 make up part of the secondary packaging containment.

Also shown in FIG. 1 is a plunger 23 and a shaft 24 disposed within the inner cylindrical bore 26 of the inner cylinder 16. The plunger 23 is shown pushed against the diaphragm 20 as if in a fired position or a position wherein the jet injector 12 is spent, as further discussed below. The position shown also depicts a ready position for filling the ampule 10 with medication, for example, by removing the cap cover 22, placing the tip into a medicine vial and moving the shaft proximally to draw in the medication. As shown, the plunger 23 is in a sealing engagement with the internal surface of the inner cylinder 16, as further discussed below.

A resilient cap seal 27 is shown compressed between the cap 22 and the diaphragm 20 at the distal end of the ampule (FIG. 1). The cap seal 27 is disposed between the cap cover 22 and the distal end of the ampule 10 and is configured to be compressed by the cap and the diaphragm to provide compliance for the nozzle 28. When the ampule 10 is filled with medication (for example at the factory), the compressed cap seal 27 prevents the medication from leaking from the inner cylinder 16 via the nozzle 28 and as well as preserves the medication's sterility by preventing contaminants from passing through the nozzle and into the medication. Alternatively, instead of utilizing the cap seal 27 to seal the nozzle 28, the present embodiment may be practiced by configuring the cap 22 with a thicker cap ridge portion 30. This cap ridge portion 30 can then contact the diaphragm 20 at about the nozzle 28 to directly compress the nozzle. The diaphragm 20, as previously discussed, is made from an elastomer and is therefore resilient in nature. The diaphragm's resiliency allows it to be compressed against the cap ridge portion 30 to thereby provide a seal for the nozzle 28, without the need for a cap seal 27.

Referring now to FIG. 2, there is shown the outer shell 18 provided in accordance with practice of the present invention. In an exemplary embodiment, the outer shell 18 is an integrally molded unit that has a cylindrical body 32, a distal end 34, and a proximal end 36. At the distal end 34, the outer shell 18 includes an end wall 38 with an end opening 40 centrally located therein. The distal end 34 also includes external threads 42 for engaging with the cap cover 22. However, instead of having external threads 42 to engage the cap, an aluminum shield can be removeably bonded to the outside surface of the end wall 38 to provide the necessary seal for the nozzle 28, which is similar to a seal in an over-the-counter medicine container.

At the proximal end 36, there is shown a set of internal threads 44 and an enlarged portion 45. The internal threads 44 are configured to engage with the threaded nipple 14 (FIG. 1) for coupling the ampule 10 to the jet injector 12. However, as further discussed below in connection with FIG. 18, instead of having the internal threads 44 at the proximal end, the outer shell 18 can include a smooth tapered sleeve. The tapered sleeve would allow the outer shell to telescopically fit over or into the distal end of the jet injector and be permanently affixed to the jet injector by either adhesive or welding. The enlarged portion 45 distal of the threads 44 is a relief point for a machine tool, which may otherwise be eliminated if the threads 44 were molded instead of machined.

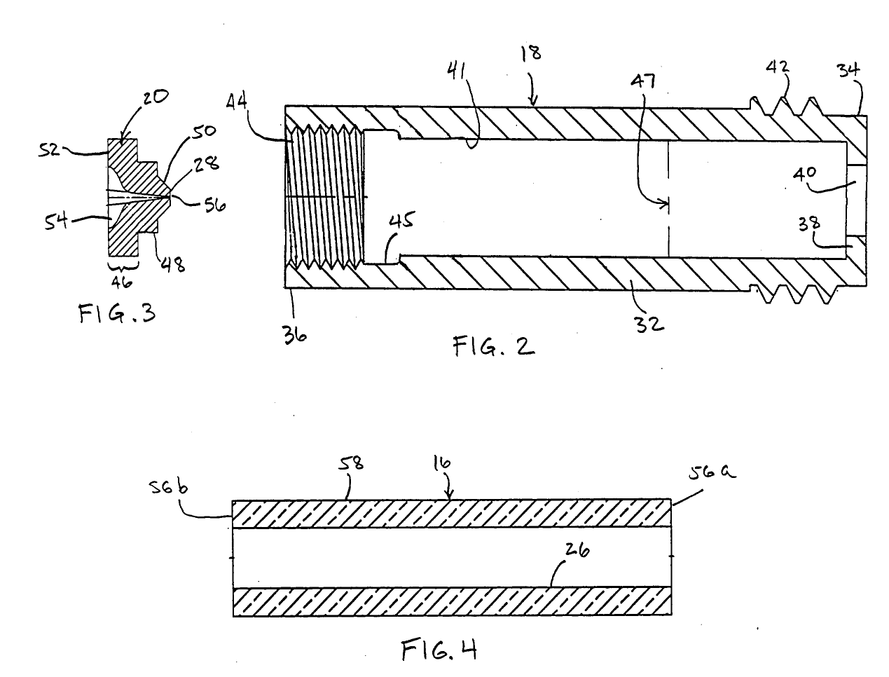

Referring now to FIG. 3, there is shown a diaphragm 20 provided in accordance with practice of the present invention. The diaphragm includes a base portion 46, a top portion 48, and a protrusion 50, which comprises the nozzle 28. The base portion 46 includes a base 52 and a cross-sectional area that is proximate the cross-sectional area 47 of the cylindrical bore 41 (FIG. 2). The top portion 48 has a cross-sectional area that is proximate the cross-sectional area of the end opening 40. The diaphragm 20 is configured to slidingly engage with the cylindrical bore 41 of the outer shell 18 and rests within the bore at the distal end of the outer shell. In this rested position, the top portion 48 is received within the end opening 40 and is seated flushed with the external end surface of the end wall 38 (FIG. 1). The protrusion 50, however, projects outward from the flushed top portion 48 to provide a distinct contact point for the nozzle 28 when placed against the skin for injection. In an exemplary embodiment, the fit between the base portion 46 and the cross-sectional area 47 of the bore 41, and between the top portion 48 and the end opening 40 of the outer shell is preferably about zero to two thousandths total clearance.

As shown in FIG. 3, the diaphragm 20 further includes a beveled cutout 54 that terminates into an orifice 56. The combination of the cutout 54 and the orifice 56 resembles a funnel and defines the nozzle 28 for discharging the medication. In an exemplary embodiment, the orifice 56 has an opening of about 0.005 to 0.010 inch, with a range of about 0.006 to 0.008 inch being more preferred.

Referring now to FIG. 4, there is shown an inner cylinder 16 provided in accordance with practice of the present invention. In an exemplary embodiment, the inner cylinder 16 is a cylindrical glass tube that has a first end 56a and a second end 56b. The inner cylinder 16 has an outer circumferential surface 58 configured to matingly abut against the cylindrical bore 41 of the outer shell 18. The inner cylinder includes the inner bore 26 for containing medication, as previously discussed. When the multi-component ampule 10 is assembled (FIG. 1), the inner cylinder 16 is configured to abut against the base 52 of the diaphragm 20 by its first end 56a and against the threaded nipple 14 by its second end 56b. The contact at the first end 56a is referred to as the first interface 62 and the contact at the second end 56b is referred to as the second interface 64. When the threaded nipple 14 is engaged and tightened against the internal threads 44, the first 62 and the second 64 interfaces are loaded and the seams defined by the interfaces are sealed from leakage (FIG. 1).

Glass is highly brittle and has a high modulus of elasticity. Glass also has a narrow proportional limit and readily fails with minimum induced strain. Thus, if a glass ampule is used with a jet injector, the glass ampule will break and will explode unless it is adequately thick. This is because a pressure of about 3,000 to 3,500 psi, and about 5,000 to 6,000 psi at the start of the injection, is generally required to administer drugs subcutaneously without a needle. In the multi-component ampule 10 provided in accordance with practice of the present invention, the inner cylinder 16, which is made from glass, is braced by the plastic outer shell 18 for reinforcement. Together, the plastic outer shell 18 and the glass inner cylinder 16 have a combined hoop strength that is sufficient to contain the pressure generated by the jet injector 12 without making the glass unnecessarily thick.

For the outer shell 18 to adequately brace or reinforce the inner cylinder 16, the slack or clearance between the cylindrical bore 26 of the outer shell and the outer circumferential surface 58 of the inner cylinder should be sufficiently tight. In an exemplary embodiment, the clearance between the outer shell 18 and the inner cylinder 16 is preferably “hand-tight”. That is, when the inner cylinder 16 is inserted into the outer shell 18, the insertion should not require tools or machines but only a force produceable by the hand. In other words, the fit between the components can be a non-interference fit. Examples of hand-tight clearance is clearance ranging from about zero to four thousandths total clearance, with about zero to two thousandths total clearance being preferred, and with about zero to one thousandths total clearance being more preferred. Examples of the inner cylinder 16 wall thickness can range from about 0.07 to 0.095 inch with 0.083 inch being more preferred. Examples of the outer shell 18 wall thickness can range from about 0.085 to 0.15 inch with 0.09 to 0.10 inch being more preferred. A person of ordinary skill in the art can appreciate that other thickness and total clearances other than the aforementioned ranges can be implemented and that these figures are exemplary only. Indeed, by changing the operating pressure, by using a glass compound, by using different plastic, by using a thinner glass and a heavier shell, etc., the fit between the inner cylinder and the outer shell and the wall thickness of the individual components can vary. Thus, such variations are contemplated to fall within the scope of the present invention.

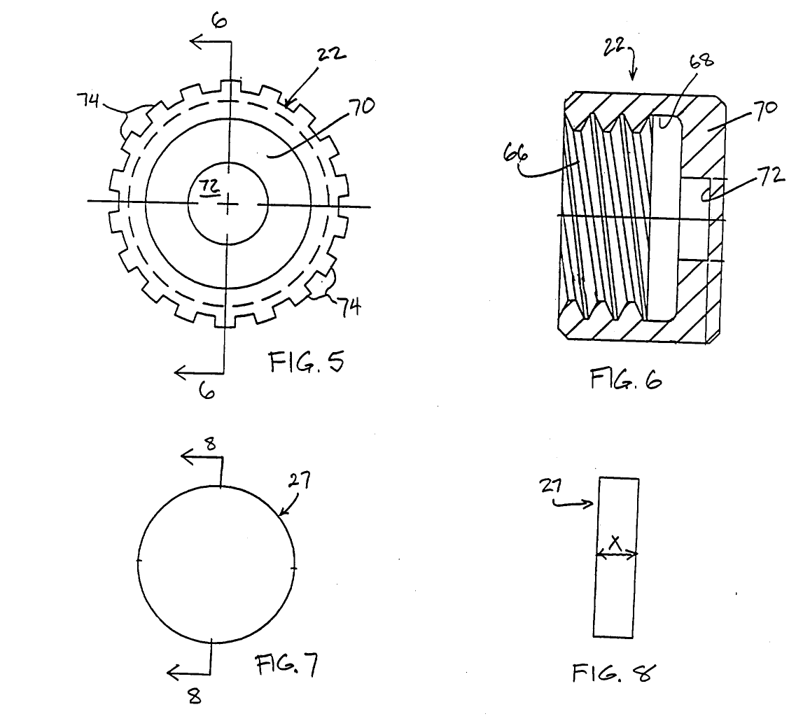

Referring now to FIGS. 5 and 6, there is shown a cap cover 22 provided in accordance with practice of the present invention. The cap cover 22 comprises internal threads 66 for threadedly engaging the external threads 42 located on the outer shell 18. The cap cover 22 includes a relief point 68 for machining the internal threads 66 but may be eliminated if the threads were molded rather than machined.

The cap cover 22 also includes an end wall 70 and a recessed portion 72 centrally disposed thereon. The recessed portion 72 is configured to receive a cap seal 27 (FIGS. 1, 7, and 8). As previously discussed, the cap seal 27 provides the necessary seal for the nozzle 28 when compressed by the cap cover 22 and the protrusion 50 located on the diaphragm 20. The cap seal 27 may be made from a number of FDA approved soft rubber or elastomer, such as silicone rubber. Still referring to FIG. 5, there is shown a series of serration members 74 circumferentially disposed along the exterior surface of the cap cover 22 for better gripping the cap cover when the same is removed. However, a smooth exterior cap surface, a dispersed array of bumps or similar gripping means may also be practiced without deviating from the scope of the invention.

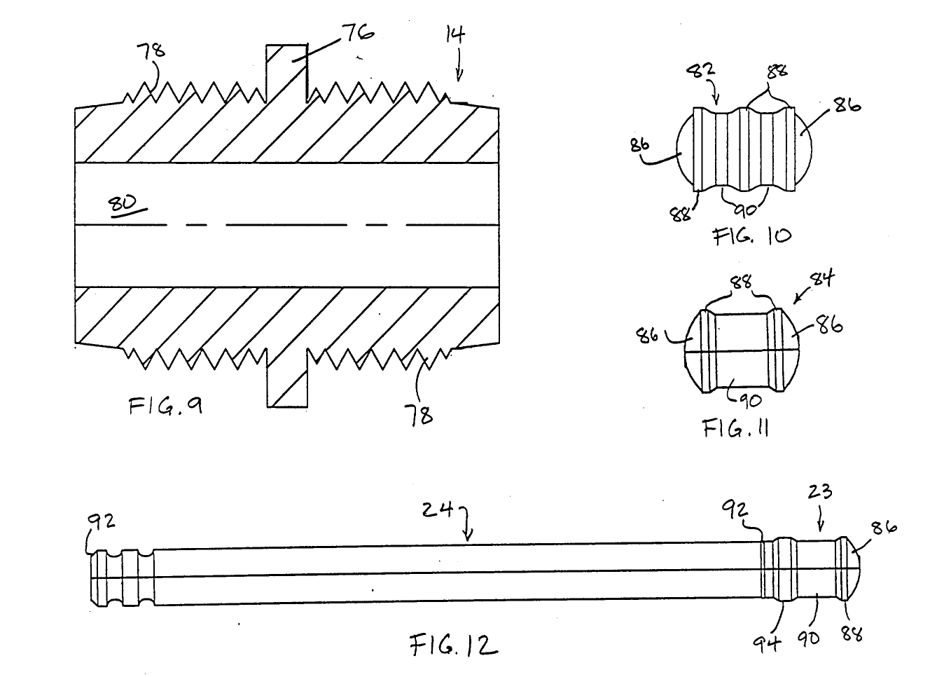

Referring now to FIGS. 7 and 8, there is shown a cap seal 27 provided in accordance with practice of the present invention. The cap seal 27 shown can be made from a variety of FDA approved elastomers or thermoplastics, such as silicone rubber and PTFE. The cap seal 27 resembles a coin in that it is circular, has a thickness X, and a cross-sectional area. The cross-sectional area is configured to fit within the recessed portion 72 located on the end wall 70 of the cap cover 22. The fit between the cap seal 27 and the recessed portion 72 is slightly interference to slightly positive clearance. The cap seal 27 is configured to be compressed by the cap cover 22 and the nozzle 28 to provide a seal for the orifice 56. Preferably, the cap seal 27 provides about 0.005 to 0.030 inch compression when compressed by the cap cover and the nozzle, with a range of about 0.008 to 0.015 inch being more preferred.

Referring now to FIG. 9, there is shown a threaded nipple 14 provided in accordance with practice of the present invention. In an exemplary embodiment, the threaded nipple 14 is symmetrical about a center flange 76 and has male threads 78 disposed on either side of the flange. The threaded nipple 14 also includes a bore 80, which acts as a channel to allow communication between the jet injector 12 and the ampule 10.

It can be appreciated that the threaded nipple 14 may be non-symmetrical and may depend on the relative dimensions of the receiving end of the jet injector 12 and the size of the proximal end 36 of the outer shell 18. For instance, the receiving end of the jet injector 12 may have a 0.5 inch threaded opening and the proximal end 36 of the outer shell 18 may have a 0.7 inch threaded opening. The threaded nipple 14 therefore will be non-symmetrical in order to accommodate the two different dimensions.

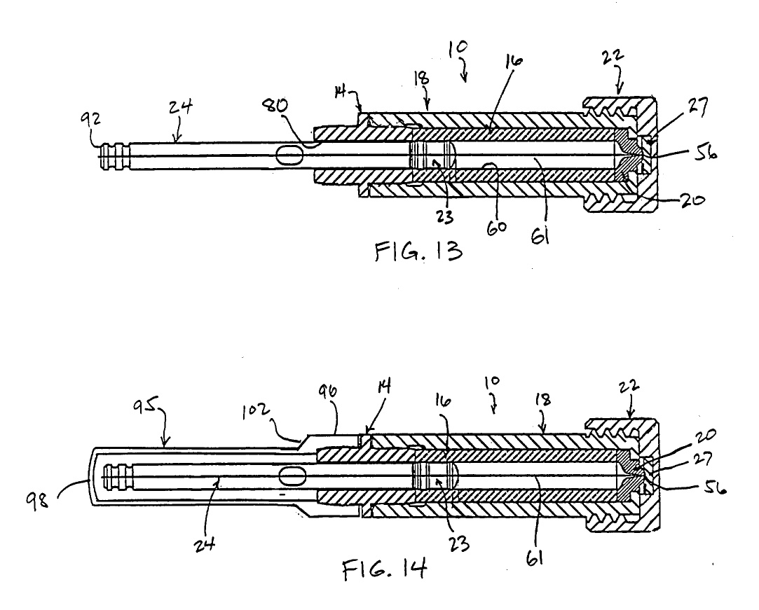

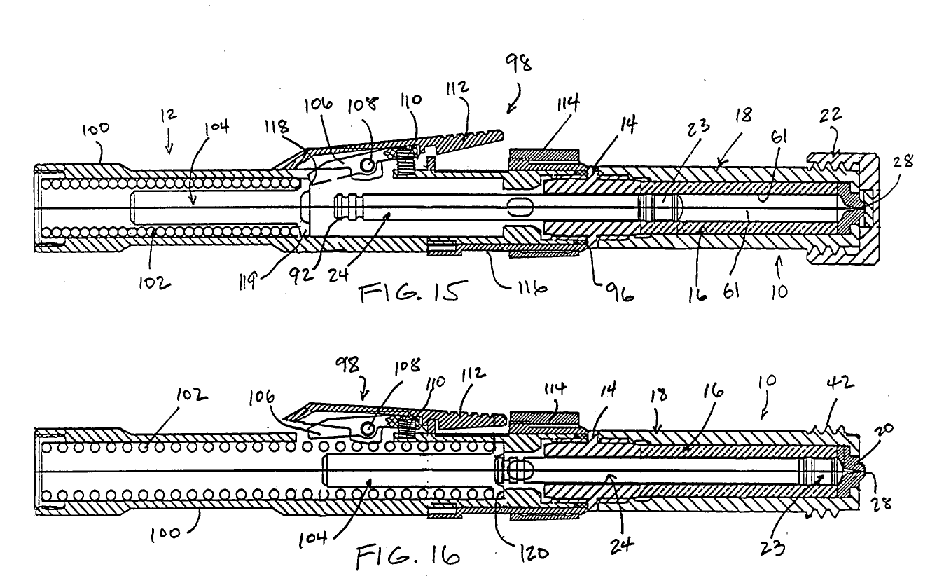

As discussed above, a plunger is configured to move from a proximal position to a distal position in the ampule 10 when the jet injector is fired to expel the medication out of the nozzle 28 (FIG. 1). The plunger moves by the action of the spring located within the jet injector, which is configured to push the piston, which then pushes the shaft, which then pushes the plunger to discharge the medication. The distal movement of the plunger compresses the medication and builds up pressure as it compresses the medication within the ampule space to deliver the necessary medication subcutaneously. The ampule space will herein be referred to as a variable medicine space, which is defined by the space between inner bore 26, the diaphragm 20, and the plunger. For reference purposes, this variable medicine space is labeled as medicine space 61 (FIGS. 13 & 14, and further discussed below). The volume defined by the variable medicine space 61 will vary depending on the location of the plunger within the ampule 10. In one exemplary embodiment, the maximum volume defined by the variable medicine space 61 is about 0.60 ml or less, with other volumes contemplated by varying the sizes of the various components of the jet injection assembly.

For pressure to adequately build within the medicine space 61 to a working pressure of about 3,000 to 3,500 psi, and about 5,000 to 6,000 psi at the start of the injection, the plunger must maintain a seal against the glass inner cylinder 16 as it travels distally in the inner bore 60 to discharge the medication out the nozzle 28. Leakage or blow-by of medication around the moving plunger should therefore be reduced to a minimum or even be eliminated as leakage will decrease the pressure build-up generated by the advancing plunger.

Referring now to FIGS. 10 and 11, shown are exemplary plungers 82, 84 provided in accordance with practice of the present invention. The exemplary plungers 82, 84 provide satisfactory sealing against the inner bore 26 of the glass cylinder 16 for building necessary operating pressure. Referring specifically to FIG. 10, the plunger 82 shown is symmetrical and includes two pusher ends 86, three marker rings 88, and two wells 90. The plunger 82 is symmetrical about the center marker ring 88 and is preferably practiced with a disposable jet injector, for reasons further discussed below. Due to its symmetrical configuration, the plunger 82 may be placed into the inner bore 26 of the glass cylinder 16 with either pusher end 86 in first. This flexibility facilitates automation by allowing a robotic machine to insert the plunger 82 into the ampule irrespective of the plunger orientation.

The plunger 84 shown in FIG. 11 is an alternative plunger. The plunger 84 is symmetrical about the center and includes two pusher ends 86, two marker rings 88, and one well 90. Both plungers 82, 84 may be made from an acceptable FDA approved elastomer such as silicone, ethylene-propylene-diene (EPDM), and the like. Alternatively, the plungers 82, 84 may have more than three marker rings, may have one or more marker rings with one or more seal rings 88 (FIG. 12) or any combination thereof.

Referring now to FIG. 12, there is shown a shaft 24 with an over-molded plunger 23 on one end and an integrally molded ribbed section 92 on the other end. The integrally molded ribbed section 92 is configured to be pushed against by a piston as the piston is propelled by a spring to launch the plunger 23 into the inner bore 26 of the glass cylinder 16 towards the diaphragm 20 located at the distal end of the ampule 20 (FIGS. 1 and 15). The plunger 23 shown is another alternative plunger which includes a pusher end 86, a receiving end 92, a marker ring 88, a seal ring 94, and a well 90. The seal ring 94 is similar to the marker ring 88 except it is slightly wider and provides more surface contact with the inner bore 26 of the inner cylinder 16. The plunger 23 is removeably connected and co-molded to the shaft as described in the '525 Ser. No. The shaft 24 may be made from a number of plastic materials such as ABS, AB, polycarbonate, PVC plastic with fiberglass injection, and the like. The shaft 24 with the over-molded plunger 23 is preferably used with the reusable injector.

FIG. 13 shows the multi-component ampule 10 in a filled position or ready position. The ampule 10 may be filled with medication in a sterile environment and packaged in the configuration shown. In the filled position, the shaft 24 is drawn proximally and extends beyond the proximal end of the nipple 14. Medicine or injectable suspension of pre-determined quantity is filled within the medicine space 61, which is the space defined by the glass inner bore 60, the diaphragm 20, and the plunger 23. In this filled position, the plunger 23 is withdrawn proximally but still remains in contact with the glass inner cylinder 16. That is, the plunger 23 does not move proximally beyond the proximal end of the inner cylinder 16, such as into the bore 80 of the nipple 14. By limiting the maximum proximal movement of the plunger 23, the medicine that is filled within the medicine space 61 is only in contact with FDA approved package materials for long term storage, which includes the inner cylinder 16, the plunger 23, and the diaphragm 20. Optionally, the exterior surface of the ampule 10 (i.e., the outer shell 18) may have markings to identify the level or volume of medication that is filled within the ampule.

The filled multi-component ampule 10 shown in FIG. 13 is configured to be received by a jet injector, such as those shown in FIGS. 15-17, shown in U.S. Pat. Nos. 5,499,972; 5,569,189; and 5,704911; and shown in Ser. No. 09/751,525. The jet injector may receive the ampule 10 by threading the nipple 14 into a threaded end of the jet injector (FIG. 15) or by permanently attaching the ampule via adhesive or welding with the jet injector (FIG. 18).

Turning to FIG. 14, there is shown a filled multi-component ampule 10, as discussed in connection with FIG. 13, with a protective housing 95 mounted to the proximal end of the threaded nipple 14. The protective housing 95 can be made from a transparent thin-walled plastic such as polyethylene or polycarbonate. The protective housing 95 includes an open base 96, an enclosed tip 93, and a threaded internal bore 97 for threadingly engaging with the nipple 14. As readily understood, the protective housing prevents the shaft 24 from being accidentally moved or bumped during packaging or shipping. In addition, the protective housing 95 also preserves the sterility of the medication by acting as an enclosure and eliminating leak source or path that can contaminate the medication. Although the protective housing 95 is shown with a tapered neck 99, the protective housing can have the shape of a uniform cylinder or any variation thereof provided it serves the aforementioned functions. The filled ampule 10 with the protective housing 95 may be packaged in the manner described in the '525 Ser. No. for shipping and storing. It is understood that the shape of the packages described in the '525 Ser. No. will have to be modified to accommodate the ampule 10 described herein.

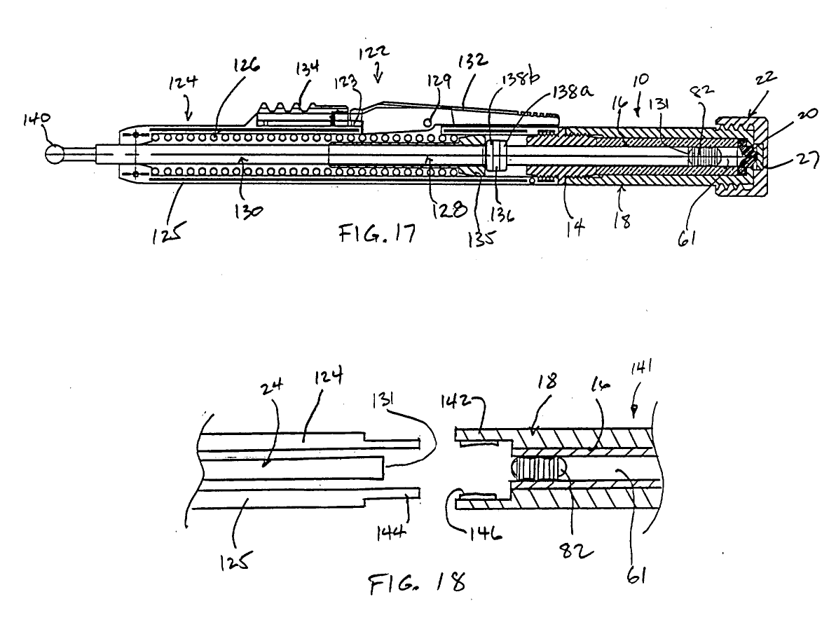

Turning to FIGS. 15 and 16, there is shown an exemplary use of the multi-component ampule 10 in accordance with practice of the present invention. In an exemplary embodiment, the filled ampule 10 (FIGS. 13 and 14) can be removed from its packaging material and assembled onto the re-useable jet injector 12 by threading the nipple 14 into the receiving end 101 of the jet injector. Once the filled ampule 10 is installed, the jet injector assembly 98 is ready for injection (FIG. 15).

The re-useable jet injector 12 shown in FIGS. 15 and 16 is substantially the same as those disclosed in the U.S. Pat. Nos. 5,499,972; 5,569,189; and 5,704,911 patents. Generally speaking, the jet injector 12 shown in FIGS. 15 and 16 includes an injector housing 100, a main spring 102 for driving a piston 104 against the shaft 24, which in turn drives the plunger 23 into the medicine space 61 to discharge the medicine out of the nozzle 28. The jet injector 12 also includes a trigger 106 that is pivoted on a pivoting pin 108 and held in an inward direction by a secondary spring 110. The trigger 106 is mechanically connected to a trigger extension arm 112, which provides the means for depressing or activating the trigger.

A safety ring 114 is provided at the distal end of the jet injector 12 for sliding engagement with a plastic sleeve 116. When the safety ring 114 is engaged, by sliding the safety ring proximally with respect to the plastic sleeve 116 until it comes to rest under the trigger extension arm 112, a downward movement by the trigger extension arm 112 about the pivoting pin 108 is delimited by the safety ring. The plastic sleeve 116 is in concentric relationship with the jet injector 12 and may be secured to the distal end of the jet injector by detents, tongue and groove means, fasteners, adhesive, or the like.

The main spring 102 is held in a compressed or cocked position by the trigger's engagement tip 118, which engages the flange 119 on the piston 104. As readily apparent, disengagement of the engagement tip 118 from the piston 104 by depressing on the trigger extension arm 112 will cause the main spring 102 to uncoil and propel the piston 104 distally. When so propelled, the piston 104 moves distally and pushes against the shaft's ribbed end 92, which pushes the shaft 24 distally. As previously discussed, this causes the plunger 23 to propel forward and compresses the medication, which then discharges out of the nozzle 28. It is understood that the cap 22 must be removed and the nozzle 28 placed against the skin of a patient before the trigger is fired for an effective delivery.

FIG. 16 shows the jet injector assembly 98 of FIG. 15 in a fired or discharged state. As shown, the trigger extension arm 112 is depressed, the main spring 102 is fully uncoiled, the piston 104 is advanced distally against a stop member 120, and the shaft 24 and plunger 23 are advanced distally toward the diaphragm 20. It can be appreciated that the plunger 23 is preferably advanced until it contacts the diaphragm 20 so that all or substantially all of the medication is discharged out of the nozzle and into the patient (FIG. 16). This maximum distal travel minimizes wastes, as certain medications are quite costly. Although the trigger extension arm 112 is shown depressed (FIG. 16), the trigger extension arm should pivot radially outward due to the secondary spring 110 (FIG. 15) immediately upon release of the trigger extension arm by the user.

Referring now to FIG. 17, there is shown an alternative multi-component ampule 10 with a disposable jet injector provided in accordance with practice of the present invention, which is generally designated 122. The jet injector assembly 122 depicted in FIG. 17 is a disposable type and resembles the type disclosed in the '525 Ser. No. The jet injector assembly 122 shown is termed disposable because once medication is dispensed following an injection, the entire jet injector assembly 122 is preferably discarded. The multi-component ampule 10 is used with the jet injector 124 by threading the nipple 14 to the receiving end of the jet injector in a similar manner as discussed with the re-useable model (FIGS. 15 and 16). The disposable jet injector 122 is shown in a fired or dispensed position.

Broadly speaking, the disposable jet injector 124 includes a housing 125, a main spring 126, a piston 128, a shaft 130, a trigger 132, and a safety device 134. The shaft further includes a gripping ball 140, a shoulder 136, and first and second cushion members 138a, 138b for limiting the distal movement of the shaft 14. This is implemented by configuring the cushion member 138a to abut the end of the nipple 14 when the shaft is propelled distally during an injection. The gripping ball 140 at the proximal end of the shaft 130 provides a gripping surface for a gripping tool (not shown) to grip and load or cock the main spring 126, as discussed in the '525 Ser. No. Subsequent to cocking the main spring 126, the gripping ball may optionally be severed from the shaft to deter recocking of the main spring.

When the shaft 130 is grasped and drawn proximally (towards the left of FIG. 17), the cushion member 138b pushes against the piston 128, at the drum portion 135 of the piston. As the shaft 130, cushion member 138b, and drum portion 135 moves proximally past the engagement tip 123 located on the trigger 132, the engagement tip 123 pivots downward, about a pivot point 129, to lock the piston and the spring 126. The contact between the engagement tip 123 and the drum portion 135 of the piston 128 maintains the spring 126 in the compressed position until the jet injector is fired (not shown). As disclosed in the '525 Ser. No., subsequent to cocking the jet injector assembly 122 by grasping and pulling on the shaft to lock the drum portion 135 against the engagement tip 123, the shaft remains freely moveable. That is, the shaft 126 still freely moves within the piston 128, as the shaft is coaxially disposed within the piston.

In an exemplary embodiment, the shaft 130 has a substantially flat distal end 131 (FIG. 17) and is not coupled to the plunger 82, which is preferably of a symmetrical type plunger. The advantage of using a symmetrical plunger is that the disposable jet injector assembly 124 is contemplated to be assembled automatically by robotic machines. Therefore, having components that are symmetrical or that are easily recognizable by the robotic machines will facilitate the automation process. Thus, the shaft distal end 131, since it does not attach to the plunger 82, can take on a number of configurations including a beveled end, a cone end, a flat end, etc.

As readily apparent, the disposable jet injector assembly 122 shown is intended to be pre-filled and packaged with medication at the factory (as the shaft 130 and the plunger 82 are not attached to provide means for filling the ampule 10). The pre-filled multi-component ampule 10 may be packaged and shipped either as two separate components (with the multi-component 10 pre-filled and separately packaged from the disposable jet injector) or pre-filled and packaged together as shown in FIG. 17. In an injection application for a separately packaged embodiment, the user simply removes the packaging material from both the disposable jet injector and the multi-component ampule and then thread the nipple 14 onto the disposable jet injector housing. Then depending on the dosage needed, the user may move the shaft 130 distally, after removing the cap 22, to release excess medication contained within the ampule.

Although shown with the symmetrical plunger (FIG. 17), the disposable jet injector assembly 122 may also be practiced with the plunger co-molded or attached to the shaft 130 and packaged with the spring pre-cocked at the factory. In this alternative application, when the end user uses the jet injector assembly 122, he or she will have to fill the multi-component ampule 10 with medication by grasping and pulling the shaft proximally to draw in medication.

FIG. 17 shows the disposable jet injector assembly 124 in a fired or discharged state (as the spring is released). Thus, it is understood that the end cap 22 should be removed from the ampule 10 before the trigger 132 is depressed to deliver the medication subcutaneously. Although FIG. 17 shows the plunger 82 spaced apart from the diaphragm, it is understood that the present embodiment is preferably practiced with the plunger 82 moved completely distally until it touches the diaphragm to thereby ensure that all of the mediation is discharged and not wasted by remaining in the medicine space 61.

Referring now to FIG. 18, there is shown an alternative interface for connecting a modified multi-component ampule 141 to the disposable jet injector 124. In the alternative embodiment, the proximal end of the outer shell 18 of the modified multi-component ampule 141 is configured to include an integrally molded coupler 142 rather than internal threads 44 as discussed with reference to FIGS. 1 and 2. The modified outer shell 18 can be filled with medication in a sterile environment and the cap 22 and the plunger 82 acting as seals to preserve the sterility of the medication.

In a corresponding fashion, the disposable jet injector 124 is configured to include an integrally molded sleeve 144. The coupler 142 on the ampule 141 is configured to fit over the sleeve 144 on the disposable jet injector 124. Once the coupler and the sleeve are mated, the interface between the two can be welded by heat or ultrasound or permanently affixed via adhesive. Still alternatively, the coupler 142 may be molded with spaced apart ridges 146 so that after the coupler is fitted with the sleeve 144, heat or ultrasonic energy may be applied to the interface region to cause the ridges to melt and to fuse the jet injector and the ampule together. When implemented, the fusion provides for a more permanent attachment.

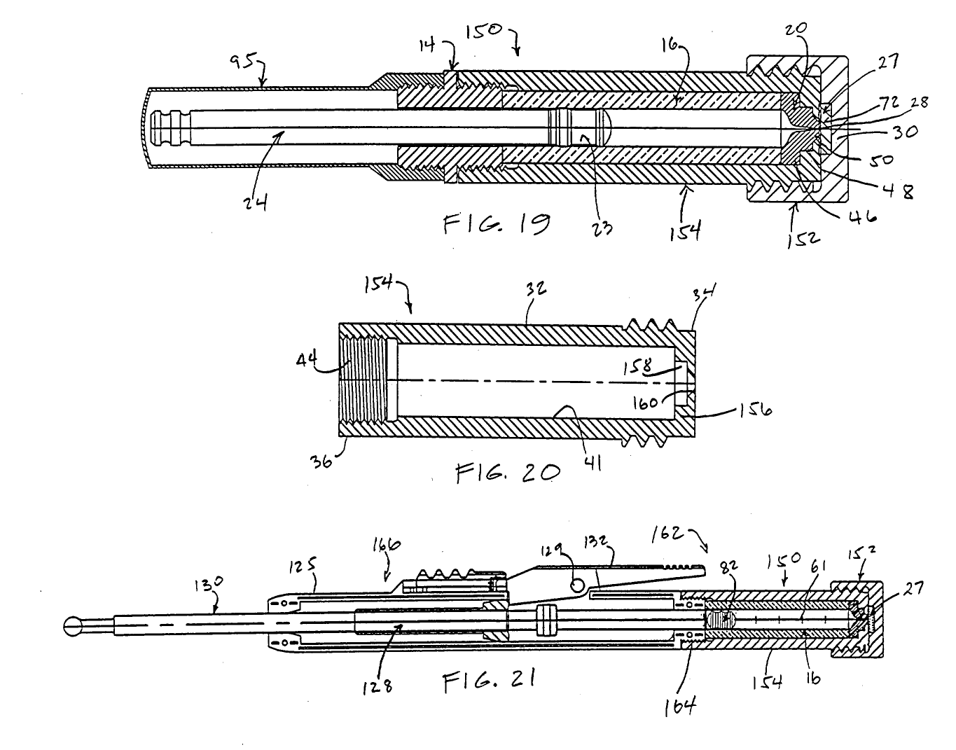

FIG. 19 shows an alternative multi-component ampule 150 having a generally flushed nozzle in a filled state with a protective housing 95 provided in accordance with practice of the present invention. The alternative multi-component ampule 150 includes essentially the same components as the multi-component ampule 10 disclosed with reference to FIGS. 1 and 14. For example, multi-component ampule 150 includes essentially the same protective housing 95, nipple 14, shaft 24, plunger 23, diaphragm 20, and inner glass cylinder 16. However, the cap cover 152 and the shell 154 have been modified to provide the multi-component ampule 150 with a flushed diaphragm 20 to outer shell configuration.

Referring specifically to FIG. 20, the modified outer shell 154 includes an end wall 156 that has a recessed portion 158 and a tapered cone section 160, which is also recessed within the end wall. The end wall 156 is configured to receive the diaphragm 20 in a flushed configuration by having a structure that corresponds to the contour of the diaphragm. For example, the tapered cone section 160 is configured to receive the diaphragm's protrusion 50, the recessed portion 158 is configured to receive the diaphragm's top portion 48, and the cylindrical bore 41 is configured to receive the diaphragm's base portion 46. As a result, the nozzle 28 located on the diaphragm 20 is positioned flushed or substantially flushed with the end exterior surface of the modified outer shell 154 (FIG. 19).

With a flushed or substantially flushed diaphragm 20 to outer shell 154 arrangement, the cap cover 152 is modified to include a relatively shallower recessed portion 72 than the cap cover 22 shown in FIGS. 1 and 6. Among other things, this modification is implemented to take up the space that is vacated by the diaphragm's protruding nozzle section 28, 50. As readily apparent, by molding the cap ridge portion 30 with a relatively thicker dimension than the same dimension shown in FIGS. 1 and 6, the same cap seal 27 may be used to provide the necessary compliance or crushed to properly seal the nozzle 28 from leak/contamination. Alternatively, the cap cover 152 can be the same as the previously described cap cover 22 (FIG. 6) but the cap seal 27 is modified to have a wider thickness X′ to provide the necessary compliance or crushed to seal the nozzle 28. Under either scenario, a cap seal 27 compression of about 0.005 to 0.030 inch is preferred, with a range of about 0.008 to 0.015 inch being more preferred.

Use of the multi-component ampule 150 shown in FIG. 19 is the same as for the multi-component ampule 10 shown with reference to FIGS. 15-17.

Referring now to FIG. 21, there is shown an alternative disposable jet injector assembly 162, which incorporates a threaded male nipple 164 into the distal end of a disposable jet injector 166 to directly couple with the multi-component ampule 150. In directly coupling the jet injector 166 with the multi-component ampule 150, the threaded nipple 14, which is used with the disposable jet injector assembly shown in FIG. 17, is eliminated. Although the jet injector 166 is shown without a spring for clarity purposes, the spring is assumed to be disposed in between the piston 128 and the housing 125, and coaxially over the shaft 130, similar to FIG. 17.

The jet injector assembly 162 is shown in a filled state, with The multi-component ampule 150 having a symmetrical plunger 82 disposed near the proximal end of the inner glass cylinder 16 and medicine contained within the medicine variable space 61. Preferably, medicine is separately pre-filled in the multi-component ampule 150, under a sterile environment, before it is assembled onto the jet injector 166. Preferably, the pre-filled multi-component ampule 150 is then assembled onto the jet injector 166 and the assembled disposable jet injector assembly 162 packaged for storage and/or shipping. Subsequent to loading the spring, the section of the shaft 130 that extends beyond and external of the housing 125 may be severed from the section of the shaft 130 positioned internally of the housing 125.

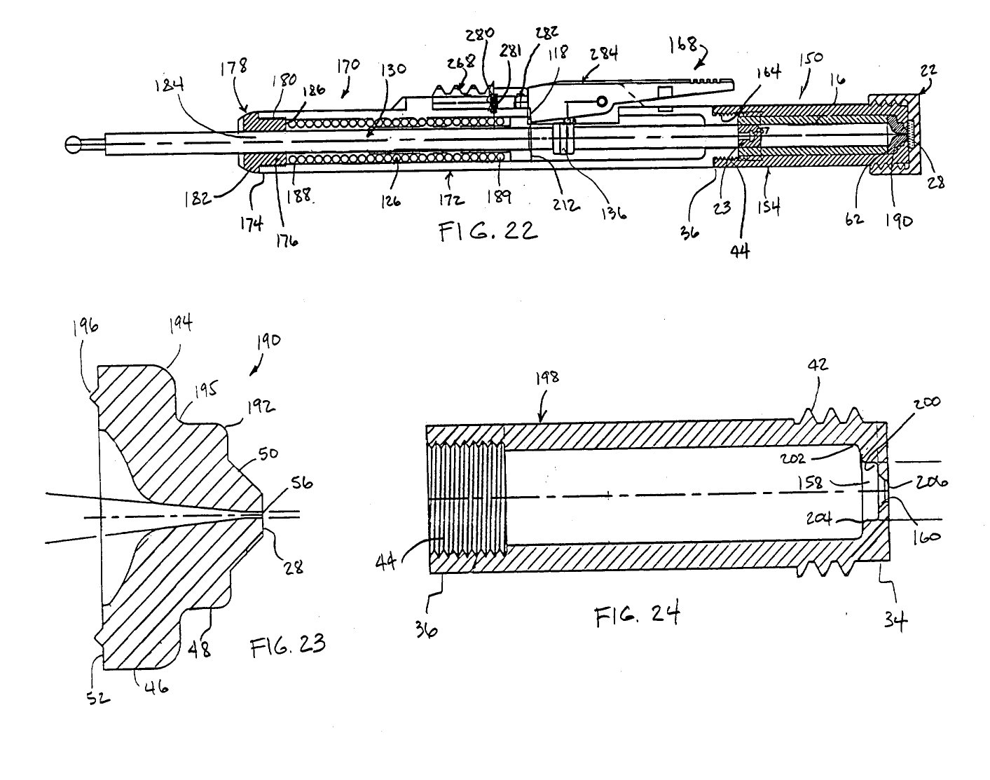

Referring now to FIG. 22, there is shown yet another jet injector assembly 168 provided in accordance with aspects of the present invention. The jet injector assembly 168 comprises a multi-component ampule 150 having an outer shell 154 comprising internal threads 44 at its proximal end 36 attached to the threaded male nipple 164 of the jet injector 170. The jet injector 170 is similar to the jet injector 124 of FIG. 17, which is similar to jet syringe disclosed in the '525 Ser. No., with a few exceptions. The housing 172 is integrally molded with an open proximal end 174 having internal threads 176. A separate plug 178 comprising a male threaded section 180 having a head section 182 is threaded to the internal threads 176 of the housing 172 to cap to the proximal end 174 of the housing 172. The plug 178 comprises a bore 184 for receiving or passing a portion of the shaft 130 therethrough.

The plug 178 comprises an end surface or support end 186 for engaging the proximal end 188 of the main spring 126. As readily apparent, a proper spring force for developing about 3,000 to 3,500 psi in the variable chamber 161, and about 5,000 to 6,000 psi at the start of the injection, may be dependent on the length of the plug 178 and how far it extends into the housing 172. For example, a spring with a higher spring constant may be required if the effective housing length is shortened by the use of the plug 178. Conversely, if the main spring 126 is the same as that shown in FIG. 17, then the housing can be lengthened to maintain an effective housing length as the length of the housing 125 shown in FIG. 17.

The distal end of the shaft 130 is shown with a co-molded plunger 23, similar to the shaft and plunger shown in FIG. 1, for use in both a pre-filled configuration or in a fill-to-use configuration. In the pre-filled configuration, injectable fluids may be filled by removing the cap cover 22, placing the nozzle 28 in fluid communication with a fluid source, such as a vial, and then aspirating fluids into the ampule 150 by moving the shaft 130 proximally (to the left of FIG. 22). An adapter or interface device for coupling the fluid source with the ampule 150 may be used to facilitate efficient filling of fluids into the ampule. Once filled to a desired level or volume, the jet injector assembly 168 may be packaged and stored for later use. As previously discussed, the portion of the shaft 130 that extends through the plug 178 and externally of the housing 172 may be severed from the remainder portion of the shaft subsequent to the filling step and prior to the packaging step. Also as previously discussed, it may be necessary to fill the ampule and package the jet injector assembly 168 in a clean room and the jet injector assembly sterilized as required by health care standards. In a fill-to-use configuration, the jet injector assembly 168 may be packaged in the configuration shown (FIG. 22). When used, the jet injector assembly 168 can be removed from its packaging and then filled with injectable fluids in the manner discussed above.

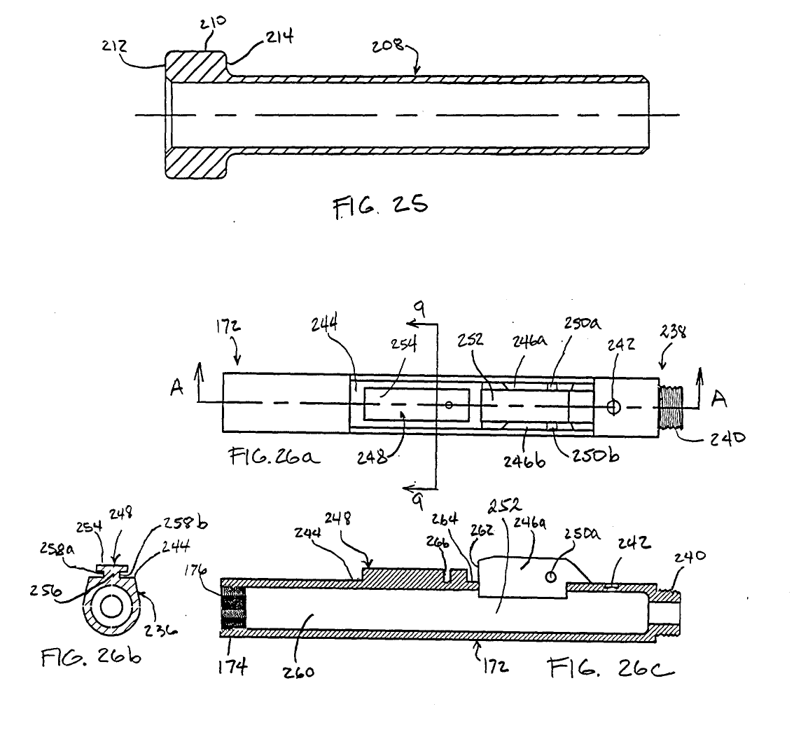

Referring now to FIG. 23, there is shown a diaphragm 190 provided in accordance with aspects of the present invention. The diaphragm 190 shown comprises a plurality of rounded contours for stress relief. For example, a first rounded contour 192 at the intersection between the protrusion 50 and the top portion 48 and a second rounded contour 194 at the intersection between the top portion 48 and the base portion 46 may be incorporated to reduce stress risers located at those intersections. A third rounded contour 195 may also be incorporated in between the first and the second contours. In addition, a protrusion 196 formed on the base 52 of the diaphragm may be incorporated to impart a high compressive load at the interface between the diaphragm 190 and the inner cylinder 16 of the multi-component ampule 150, at the first interface 62 (FIG. 22). The protrusion 196 may comprise a circular ring having thickness of about five thousandths to twenty thousandths when measured from the base 52.

The diaphragm 190 may be made from an elastomer, such as silicone rubber and used with anyone of the multi-component ampules discussed above, provided that the shells in those ampules are modified to mate with the rounded contours of the diaphragm. Alternatively, cyclic olefin copolymers, polyethylene-pentene, teflon, polypropylene, and other materials having comparable water absorption values may also be used without deviating from the scope of the present invention. Preferably, the selected material should have a Durometer A rating of greater than about 40.

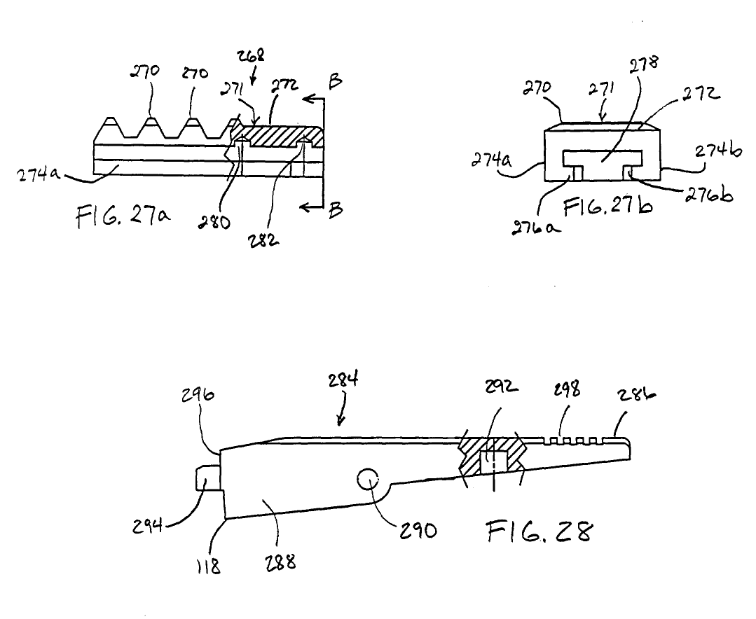

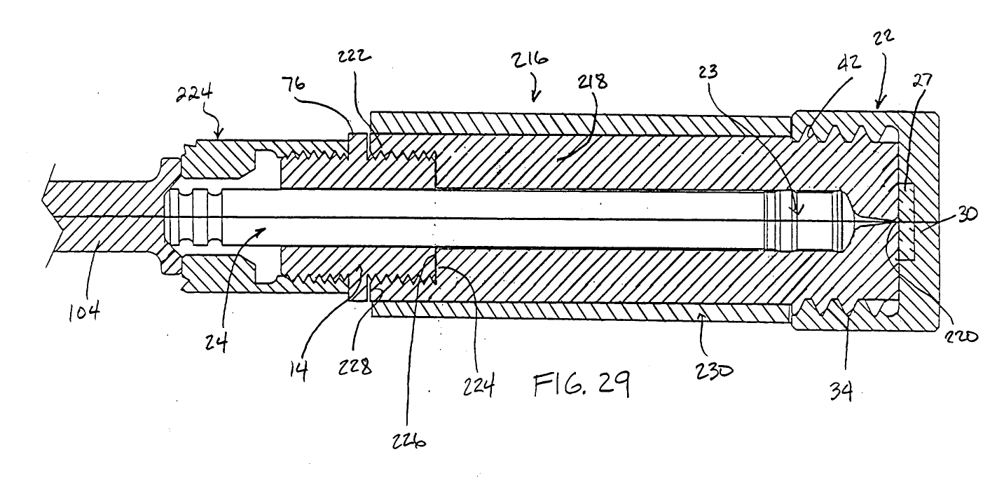

Referring now to FIG. 24, an outer shell 198 comprising corresponding rounded contours for receiving the diaphragm 190 of FIG. 23 is shown. The outer shell 198 is similar to the outer shell 154 shown in FIG. 20 with a few modifications. In particular, a first rounded contour 200, a second rounded contour 202, and a third rounded contour 204 may be incorporated at the distal end 34 of the outer shell 198 to form a one-on-one fit with the rounded contours 192, 194, 195 of the diaphragm 190. The rounded contours of both the outer shell and the diaphragm preferably comprise a bending radius of about 0.01 inch to 1 inch. However, other bending radii may be incorporated without deviating from the scope of the present invention.

Still referring to FIG. 24, a generally leveled opening 206 is provided at the distal end of the tapered cone section 160 of the outer shell 198. The leveled opening 206 comprises wall surfaces that are generally parallel to the cylindrical wall sections of the outer shell. The leveled opening 206 is positioned just distal of the sloped section 206 and provides clearance for the nozzle 28 of the diaphragm 190 to expand when fluids pass through the opening 56 thereof.

Although the outer shell 198 is shown with external threads 42 at its distal end 34 to receive a cap 22 (See, e.g., FIG. 1), the threads may be eliminated if a foil innerseal is used to seal the orifice 28 of the diaphragm, as previously discussed. Still alternatively, aluminum seal flip off or tear off caps may be used to seal the orifice 28 from contamination. Such flip off or tear off caps are commercially available from Kimble Kontes and from Wheaton Science Products. Exemplary foil innerseals useable with the multi-component ampules of the present invention include those made from Selig Sealing Products, Inc., of Oakbrook Terrace, Ill., and disclosed in U.S. Pat. Nos. 5,702,015; 5,860,544, 5,915,577; and 6,461,714, and their equivalents. The contents of these patents are incorporated herein by reference.

Referring now to FIG. 25, there is shown an alternative piston 208 provided in accordance with aspects of the present invention. As shown, the piston 208 comprises a drum 210, a first drum surface 212 and a second drum surface 214. The piston 208 may be useable with the jet syringe of FIGS. 17, 21, or 22. When incorporated, the first drum surface 212 is adapted to abut the shoulder 136 of the shaft 130, and the second drum surface 214 is adapted to abut the distal end 189 of the main spring 126 (See, e.g., FIG. 22). The shoulder of the shaft may include cushion members located on either side of the shoulder. In one embodiment, the first drum surface 212 comprises a surface section and the second drum surface 214 comprises a surface section that are parallel to one another. The piston preferably comprises a die cast zinc, copper and aluminum alloy. The proportions of zinc, copper and aluminum are selected to provide the mass necessary to eject fluid from an ampule with the necessary force as to effect a subcutaneous injection. The use of such an alloy provides the necessary mass to drive the shaft in a manner that assures proper operation of the present invention, e.g., the development of pressure within the ampule of approximately 3,000-3,500 psi. The use of this alloy also reduces costs sufficiently to facilitate the construction of a disposable device. However, other alloys and even non-metallic materials may be used provided they satisfy the mass necessary to propel the shaft.

Referring now to FIG. 26a, there is shown a top plan view of a jet syringe housing 172 provided in accordance with aspects of the present invention. The housing 172 may be integrally molded with the features to be discussed below and usable with the plug 178 of FIG. 22.

At the distal end 238 of the housing 172, an integrally molded male nipple 240 is provided to engage with a female coupler of a multi-component ampule, such as one of the ampules shown in FIGS. 1, 19, and 29. However, as readily apparent to a person of ordinary skill in the art, a female coupler may be incorporated with the housing 172 of FIG. 26a if the multi-component ampule comprises a male nipple. A secondary spring positioner 242 for receiving a secondary spring (See, e.g., secondary spring 110 of FIG. 15) is positioned just proximal of the male nipple 240. The positioner 242 generally comprises a circular opening molded into the housing 172 for receiving an end of the secondary spring, which is used to bias the trigger radially outwardly in a ready to fire position.

A generally flat mounting plane 244 is located just proximal of the positioner 242. The mounting plane 244 resembles a generally flat surface area provided on a generally cylindrical surface section of the housing 172. The mounting plane 244 comprises means for assembling a trigger and a trigger safety slide. In one exemplary embodiment, the mounting plane 244 comprises a pair of retaining walls 246a, 246b for engaging a trigger and a safety channel 248 for sliding engagement with a trigger safety slide, as further discussed below. Each retaining wall includes a boss or aperture 250a, 250b for receiving a pin to pivotally mount the trigger. A generally rectangular opening 252 on the mounting plane 244 in between the retaining walls 246a, 246b is provided to receive the engagement tip portion of a trigger, as further discussed below. The rectangular opening 252 is sized to provide sufficient space for accommodating the engagement tip portion of the trigger.

A safety channel 248 is positioned just proximal of the rectangular opening 252. The safety channel 248 includes a radial wall (not shown) that extends from the mounting plane 244 and a channel plate 254 positioned on the radial wall. Referring to FIG. 26b, which is a cross-sectional view of the housing of FIG. 26a taken at line 9-9, the safety channel 248 resembles a “T”, with the radial wall 256 resembling the vertical portion of the T and the channel plate 254 resembling the horizontal portion of the T. The radial wall 256 and the channel plate 254 defines two grooves 258a, 258b for engaging with two tongues located on the trigger safety slide, as further discussed below.

FIG. 26c is a cross-sectional side view of the housing 172 of FIG. 26a taken along line A-A. A threaded coupler or female threads 176 is provided with the housing 172 at the proximal end 174 thereof for engaging a plug, such as the plug 178 of FIG. 22. Adhesive or welding may be incorporated to permanently secure the plug 178 to the housing 172.

A wall stop 262 is shown at the proximal end of the retaining wall 246a, adjacent the safety channel 248. The wall stop 262 is provided to stop the distal movement of a safety slide (not shown) when the same is mounted over the safety channel 248. A trigger landing 264 is located just proximal of the wall stop 262 and comprises a part of the mounting plane 244. As further discussed below, the trigger landing 264 provides a rest surface for a- trigger extension tab (element 294 of FIG. 28). The trigger extension tab is configured to abut against a portion of the trigger safety slide to delimit movement of the trigger and hence prevent the trigger from activating the spring mechanism assembly. A generally cylindrical retaining bore 266 may be provided on the safety channel 248. When incorporated, a pin may be inserted therein so that the pin engages an underside of the trigger safety slide to lock the trigger safety slide. The pin then functions to keep the safety slide from sliding off of the trigger extension tab or the safety channel 248.

A semi-schematic partial cut-away side view of a trigger safety slide 268 provided in accordance with aspects of the present invention is shown in FIG. 26a. The safety slide 268 comprises a plurality of raised gripping members 270 on the top surface 271 of the safety slide for facilitating gripping manipulating moving the safety slide off of the trigger. The raised gripping members 270 resemble elongated pyramids but can comprise an array of bumps or any raised features for enhancing gripping. Still alternatively, a flat finish may also be incorporated as a user can simply apply a greater downward force on the safety slide 268 to slide the same off of the trigger.

A safety cover portion 272 is located on the top surface 271 just distal of the gripping members 270. The safety cover portion 272 is adapted to slide over the trigger extension tab of the trigger to prevent the tab, and hence the trigger, from lifting and discharging the spring mechanism. Two side walls 274a, 274b extend downwardly from the top surface 271 and two engagement walls 276a, 276b (FIG. 26b) extend inwardly at the end thereof to define a sliding receiving space 278. The two engagement walls 276a, 276b function as tongues to engage with the grooves 258a, 258b of the safety channel 248 located on the housing.

A lock bore 280 and a retract bore 282 are formed on an underside of the top surface 271. The lock bore 280 is adapted to engage with a pin, which is secured to the cylinder retaining bore 266 of the safety channel 248 of the housing 172. The pin fixes the safety slide 268 over the trigger extension tab of the trigger to set and secure the safety slide in a lock position. When the safety slide 268 is unlatched or taken off of safety, the proximal movement of the safety slide disengages the lock bore 280 from the pin 281 and moves the retract bore 282 over the pin to engage with the pin 281 (See, e.g., FIG. 22).

FIG. 26b is a semi-schematic end view of the safety slide 268 of FIG. 26a taken at line B-B. As shown, the sliding receiving space 278 is defined by the top surface 271, the side walls 274a, 274b, and the engagement walls 276a, 276b. The sliding receiving space 278 resembles a “T”, which is a negative cavity of the safety channel 248 of FIG. 26b.

Referring now to FIG. 28, there is shown a semi-schematic partial cut away view of a trigger 284 provided in accordance with aspects of the present invention. The trigger 284 comprises a triggering section 286, for pressing and triggering the spring mechanism, and a trigger set section 288, which comprises an engagement tip 118 for engaging the drum 210 of a piston 208 (See, e.g., FIG. 25). The trigger 284 further comprises a pivoting bore 290 for pivoting engagement with the bores 250a, 250b of the retaining walls 246a, 246b of the housing 172 via a pivot pin (not shown). The trigger may be molded from a thermoplastic material or machined from a metal stock.

A secondary spring positioner 292 is located distal of the pivoting bore 290 for receiving a secondary spring (See, e.g., spring 110 of FIG. 15). The positioner 292 cooperates with the secondary spring positioner 242 of the housing 172 and the secondary spring to bias the triggering section 286 of the trigger 284 radially outwardly away from the housing. This bias configuration facilitates engagement between the engagement tip 118 and the drum 210 of the piston 208 when the spring mechanism is cocked.

A trigger extension tab 294 extends from a proximal end wall 296 of the trigger 284. As previously discussed, the trigger extension tab 294 is configured to rest on the trigger landing 164 of the housing and held by the safety cover portion 272 of the safety slide 268 to prevent the trigger 284 from triggering. A plurality of gripping members 298 may also be incorporated on the trigger to facilitate gripping by a user and to connote an point of activation.

Referring again to FIG. 22, the trigger 284 and the safety slide 268 may be assembled onto the housing 172 as shown. In particular, the safety slide 268 is shown in the safety position and abutting the trigger extension tab 294 to prevent pivoting of the trigger 284. In the configuration shown, the engagement tip 118 of the trigger 284 is abutted against the first drum surface 212 of the piston 208 when the main spring 126 is cocked. As previously discussed, the shaft 130, which is coaxially disposed with the piston 208, remains freely moveable so that injectable fluids may be aspirated into the cavity of the multi-component ampule 150.

Referring now to FIG. 29, there is shown an alternative multi-component ampule 216 provided in accordance with aspects of the present invention. The ampule shown includes an inner shell 218 having an integrally molded nozzle 220, which defines an orifice. The inner shell 218 comprises a female coupler 222 for threaded engagement with a threaded nipple 14, which then connects to a jet syringe 224 (partially shown). The jet syringe can be any one of the various jet syringes discussed above. Alternatively, the inner shell 218 can couple directly to a threaded male nipple of a jet syringe without the threaded nipple 14, such as coupling with the threaded male nipple 164 of the jet syringe 166 of FIG. 21. Preferably, the coupler 222 comprises a sufficient depth so that the coupler base 224 abuts the male end 226 of the threaded nipple 14 before the coupler end 228 abut the center flange 76 of the male nipple. Optionally, an 0-ring or a gasket may be used at the interface between the male end 226 of the male nipple and the coupler base 224 of the female coupler 222.

Male threads 42 may be formed at the exterior distal end of the inner shell 218 to threadingly engage with a cap cover 22. A cap seal 27 comprising an elastomer may be placed between the nozzle 220 and the cap ridge portion 30 of the cap 22 to provide the desired seal for the nozzle 220. However, as previously discussed, a foil innerseal may be used instead of a cap cover to seal the nozzle 220. If the foil innerseal is implemented, the male threads 42 may be eliminated and the inner shell 218 resembles a generally cylindrical shell, which may include a slight taper or a draft angle for molding purposes.

The inner shell 218 is preferably made from a low moisture permeable thermoplastic having long term drug storage capability. Suitable plastic includes cyclic olefin copolymers, polyehtylene-pentene, and glass-clear polypropylene. The inner shell 218 preferably comprises an inner diameter and an outside diameter, i.e., wall thickness, sized to operate with an operating pressure of about 5,000 to 6,000 psi at the beginning of a jet syringe discharge and about 3,000 to 3,500 psi nominal operating pressure.

An optional outer thermoplastic shell 230 may be co-molded or over-molded with the inner shell 218 to reinforce the inner shell and/or to provide an oxygen impermeable barrier for the inner shell. The outer shell 230 may be made from polyethlene terephthalate (PET), ethylene vinyl alcohol (EVOH), polyvinylidene chloride (PVDC), polycarbonate or their equivalents. These materials share similar oxygen permeability characteristic. The outer shell 230 should have a shell length sufficient to extend from the coupler end 228 to the male threads 42, when a cap cover 22 is used. If a foil innerseal is used, the outer shell 230 should extend the full length of the inner shell 218. Alternatively, instead of co-molding the outer shell 230 over the inner shell 218, the outer shell can be slid over the inner shell and the clearance between the two comprising a total clearance of about zero to three thousandths.

A shaft 24 with a plunger 23 is shown occupying the cavity of the inner shell 218. However, the present multi-component ampule 216 may be implemented with the plunger shown in FIGS. 10 and 11 for a pre-filled configuration or with a co-molded plunger of FIG. 22.

Although the preferred embodiments of the invention have been described with some specificity, the description and drawings set forth herein are not intended to be delimiting, and persons of ordinary skill in the art will understand that various modifications may be made to the embodiments discussed herein without departing from the scope of the invention, and all such changes and modifications are intended to be encompassed within the appended claims. Various changes to the ampule may be made including manufacturing the dimensions differently, using different FDA approved materials, changing the tolerances, using tempered glass, etc. Other example of changes may include modifying the way the ampule is connected to the jet injector, the way the shaft and the piston are shaped/configured, and the way the plunger is shaped/configured. Accordingly, many alterations and modifications may be made by those having ordinary skill in the art without deviating from the spirit and scope of the invention.

What is claimed is:

1. A jet injector assembly comprising an ampule having an inner shell and an outer shell attached to a spring injector, wherein the spring injector comprising a housing comprising a plug having a passage engaged to an end of the housing, a shaft coaxially disposed with a piston and with a spring and partially extending through the passage of the plug, and wherein the spring is in abutting relationship with the piston and the plug.

2. The jet injector assembly of claim 1, wherein the inner shell comprises a glass material.

3. The jet injector assembly of claim 1, wherein the inner shell comprises cyclic olefin copolymers or polyethylene-pentene.

4. The jet injector assembly of claim 1, further comprising a nozzle comprising an orifice integrally molded with the inner shell.

5. The jet injector assembly of claim 1, further comprising a diaphragm comprising a nozzle and an orifice, and wherein the diaphragm is positioned adjacent a distal end wall of the inner shell.

6. The jet injector assembly of claim 1, wherein the housing comprises a first end having a male threaded plug and a second end having a female threaded coupler.

7. The jet injector assembly of claim 1, wherein the housing comprises a safety channel comprising two grooves for receiving a safety slide.

8. The jet injector assembly of claim 1, further comprising a plurality of walls extending radially from a surface of the housing, the plurality of walls each comprising a bore for receiving a pivot pin.

9. The jet injector assembly of claim 1, wherein the housing comprises two female threaded couplers at its two ends and wherein one of the ends is connected to a threaded nipple.

10. The jet injector assembly of claim 1, further comprising a trigger comprising a triggering set section comprising a trigger engagement tip, wherein the trigger engagement tip is abutted against the piston and the piston compresses the spring.

11. The jet injector assembly of claim 1, wherein the shaft comprises a shoulder and the shoulder is abutted directly or indirectly against a portion of the piston.

12. The jet injector assembly of claim 1, wherein the outer shell comprises a transparent thermoplastic material.

13. The jet injector assembly of claim 1, wherein the plug is threadedly engaged with the end of the housing.

14. The jet injector assembly of claim 1, wherein the outer shell is threadedly engaged to the spring injector assembly.

15. The jet injector assembly of claim 1, wherein the inner shell is threadedly engaged to the spring injector assembly.

16. The jet injector assembly of claim 5, wherein the diaphragm comprises an protrusion on a base section, and wherein the protrusion is abutted against an end of the inner shell.

17. The jet injector assembly of claim 1, wherein a foil innerseal is adhered to an end surface of the inner shell.

18. The jet injector assembly of claim 1, wherein a foil innerseal is adhered to an end surface of the outer shell.

19. The jet injector assembly of claim 1, further comprising volumetric markings on the ampule.

20. The jet injector assembly of claim 1, wherein the jet injector assembly is discarded after a single injection.

21. A jet injector assembly comprising:
an ampule comprising: an inner shell having a first closed end comprising an integrally molded nozzle and an open second end comprising female threads, wherein the inner shell is made from a first thermoplastic material; and an outer shell co-molded to the inner shell having first and second open ends, wherein the outer shell is made from a second thermoplastic material;
a plunger in dynamic sealing communication with an interior cavity of the inner shell, wherein the plunger is adapted to discharge contents from within the interior cavity of the inner shell out of the integrally molded nozzle when moved from a first position to a second position within the inner shell;
a cap cover or a foil innerseal attached to a distal end of the inner shell; and
wherein the female threads are engaged to male threads of a jet injector or to male threads of a nipple and wherein the nipple is engaged to the jet injector.

22. The jet injector assembly of claim 21, wherein the inner shell is made from cyclic olefin copolymers.

23. The jet injector assembly of claim 21, wherein the outer shell is made from PET, EVOH, PVDC, or polycarbonate.

24. The jet injector assembly of claim 21, wherein the jet injector comprises a housing and a spring mechanism disposed inside the housing.

25. The jet injector assembly of claim 21, wherein the jet injector assembly comprises a shaft coaxially disposed with a piston and with a spring.

26. The jet injector assembly of claim 21, wherein the jet injector assembly comprises a housing and wherein the housing is made from glass loaded acrylonitrile-butadiene-styrene (ABS).

27. The jet injector assembly of claim 21, wherein the jet injector assembly comprises housing and a trigger pivotally attached to two radially extending walls extending from the housing.

28. A jet injector assembly comprising an ampule attached to a jet injector; wherein the ampule comprises an inner shell made from a first material having a first end, a second end, and an interior cavity; a diaphragm made from a second material positioned in the interior cavity of the inner shell adjacent the first end; and an outer shell made from a third material coaxially disposed over the inner shell.

29. The jet injector assembly of claim 28, wherein the diaphragm comprises a base portion, a top portion, and a protrusion comprising a nozzle and the nozzle defining an orifice.

30. The jet injector assembly of claim 29, wherein the base portion comprises a raised protrusion.

31. The jet injector assembly of claim 29, wherein the to pop portion and the base portion comprise a plurality of rounded contours.

32. The jet injector assembly of claim 29, wherein the first end of the inner shell comprises an end wall and an opening, and wherein the protrusion on the diaphragm is positioned proximate the opening.

33. The jet injector assembly of claim 28, wherein the first material is glass, the second material is thermoplastic, and the third material is one of silicone, cyclic olefin copolymers, or teflon.

34. The jet injector assembly of claim 33, wherein the glass is tempered glass.

35. The jet injector assembly of claim 28, wherein the ampule is threadedly attached to the jet injector.

36. The jet injector assembly of claim 28, wherein the jet injector comprises a spring mechanism disposed inside a housing.

37. The jet injector assembly of claim 28, further comprising a plunger in dynamic sealing communication with the interior cavity of the inner shell.

38. The jet injector assembly of claim 28, wherein the jet injector comprises a steel housing and a spring mechanism disposed inside the steel housing, wherein the spring mechanism comprises a piston and a spring.

39. The jet injector assembly of claim 28, wherein a foil innerseal or a cap cover is attached an exterior surface of the outer shell.
